SEQUENCE LISTING (2 sequences)

SEQ ID NO: 1 - RNA, 22 nt, Homo sapiens
uggcagugucuuagcugguugu

SEQ ID NO: 2 - RNA, 22 nt, Caenorhabditis elegans
ucaccggguguacaucagcuaa

(19) United States
(12) Patent Application Publication
(10) Pub. No.: US 2022/0340962 A1
(43) Pub. Date: Oct. 27, 2022

(54) QUANTITATIVE DETECTION OF MICRO-RNAS

(71) Applicant: GENFIT, Loos (FR)

(72) Inventor: ZOUHER MAJD, ENNETIERES EN WEPPES (FR)

(21) Appl. No.: 17/237,789

(22) Filed: Apr. 22, 2021

(51) Int. Cl. C12Q 1/6851

(52) U.S. Cl. CPC C12Q 1/6851; C12Q 2600/178

(57) ABSTRACT

The present invention is directed to novel compositions and methods useful for the quantitative detection of microRNAs. More particularly, it relates to premixes useful in reverse transcriptase-quantitative polymerase chain reaction (RT-qPCR) reactions for the detection of miR-34.

### REFERENCE TO SEQUENCE LISTING

The Sequence Listing for this application is labeled “Seq-List.txt” which was created on Jun. 21, 2021 and is 1 KB.

### FIELD OF THE INVENTION

The present invention is directed to novel compositions and methods useful for the quantitative detection of microRNAs. More particularly, it relates to premixes useful in reverse transcriptase-quantitative polymerase chain reaction (RT-qPCR) reactions for the detection of miR-34.

### BACKGROUND OF THE INVENTION

MicroRNAs (miRNAs) are a family of endogenous, small, noncoding functional RNAs inhibiting the translation of proteins by binding to 3′-UTR of the target mRNA. They were also found to be useful circulating biomarkers. For example, the level of hsa-miR-34a-5p is correlated to nonalcoholic fatty liver disease (NAFLD) and nonalcoholic steatohepatitis (NASH). Therefore, accurate means for the quantitative detection of miRNAs are needed. Reverse transcriptase quantitative polymerase chain reaction (RT-qPCR) is the most widely used method to quantitatively detect miRNAs. It comprises a reverse transcription (RT) step, to produce a cDNA from the miRNA potentially present in a test sample, and a quantitative polymerase chain reaction (qPCR) step, to amplify the cDNA generated in the preceding step.

Current standard RT and qPCR assay procedures require many steps of pipetting and mixing for reagent preparation and distribution. Typically, 10 steps are required for the RT reaction, and 4 steps are required for the qPCR reaction. These many pipetting steps and required sample handling increase the risk for errors, such as the generation of false-positives during the amplification process, or the absence of detection which can happen if a RNAse is inadvertently introduced into the reaction mix during one of the multiple steps involved in these methods. It is important to prevent those errors to happen, in particular in the context of diagnosis where accurate quantitation of a marker is required. Moreover, reducing pipetting steps would also reduce the operator's hands-on time.

### BRIEF DESCRIPTION OF THE FIGURES

It is herein provided compositions and methods to solve these problems.

FIG. 1: schematic view of the different RT premixes tested.

FIG. 2: schematic view of the different qPCR premixes tested.

FIGS. 3 to 5: The four RT premixes are tested using miR34a positive controls (C1 High, C2 medium and C3 low) after one month at −20° C. and compared to the classical preparation.

FIGS. 6 to 8: The four RT premixes are tested using miR34a positive controls (C1 High, C2 medium and C3 low) after 6 months at −20° C. and compared to the classical preparation.

FIGS. 9 to 11: The four qPCR premixes are tested using positive miR34a positive controls (C1 High, C2 medium and C3 low) after one month at −20° C. and compared to the classical preparation.

FIGS. 12 to 14: The four qPCR premixes are tested using positive miR34a positive controls (C1 High, C2 medium and C3 low) after 6 months at −20° C. and compared to the classical preparation.

### DESCRIPTION OF THE INVENTION

The present invention relates to compositions useful in the practice of a RT-qPCR method for detecting a miRNA, preferably hsa-miR-34a-5p.

Reverse Transcriptase Premix

A first aspect of the invention relates to a first composition, hereinafter referred to as a “RT premix”. The RT premix of the invention comprises, or consists of:
a deoxyribonucleotide trisphosphate (dNTP) mix containing dATP, dTTP, dCTP and dGTP;
a sequence-specific primer suitable to specifically prime the reverse transcription of the miRNA of interest to be quantitatively detected in a test sample;
a sequence-specific primer suitable to specifically prime the reverse transcription of a control miRNA, useful to normalize the quantity of the miRNA of interest;
a reverse transcription buffer;
Tris-EDTA buffer; and
nuclease-free water.

In a particular embodiment, each dNTP of the dNTP mix is at a concentration of 25 mM before addition to the premix. In yet another embodiment, each dNTP is at a final concentration comprised between 500 and 1000 μM in the RT premix. In a particular embodiment, each dNTP is at a final concentration of 850 μM in the RT premix.

In a particular embodiment, the miRNA of interest is miR-34, in particular hsa-miR-34a-5p whose nucleic acid sequence consists of 5'-UGGCAGUGUCUUAGCUGGUUGU-3' (SEQ ID NO: 1) (accession number in the miRBase: MIMAT0000255). Primers useful for use in a RT reaction specific of hsa-miR-34a-5p are known in the art. For example, one skilled in the art can use the primer of the TaqMan MicroRNA assay, kit number 4440888 (Applied Biosystems).

In a particular embodiment, the control miRNA is an endogenous housekeeping miRNA (i.e. a stably expressed miRNA), or a spiked-in, synthetic miRNA (i.e. a miRNA that is added in a defined amount to the test sample before the RT reaction). In a particular embodiment, the spike-in synthetic miRNA is a non-human miRNA, an exogenous miRNA absent in the subject sample. For example, the non-human synthetic or exogenous miRNA can be derived from Caenorhabditis elegans (cel) or Arabidopsis thaliana (ath). In a particular embodiment, the spiked-in miRNA is cel-miR40 (in particular cel-miR-40-3p) or cel-miR-39 (in particular cel-miR-39-3p). Other non-human miRNA for use as synthetic miRNA in the context of the present invention include, without limitation, ath-miR-159a and cel-miR-40-3p. In a particular embodiment, the spiked-in miRNA is cel-miR-40-3p whose nucleic acid sequence consists of 5′-UCACCGGGUGUACAUCAGCUAA-3′ (SEQ ID NO: 2)(accession number in the miRBase: MI0000011). It will be understood that defined quantities of cel-miR-40-3p can be introduced in a test sample, to then serve as a control miRNA useful in normalizing the detected quantity of the miRNA of interest, such as to normalize the detected quantity of hsa-miR-34a-5p. Primers useful for use in a RT reaction specific of cel-miR-40-3p are known in the art. For example, one skilled in the art can use the primer of the TaqMan MicroRNA assay, kit number CCU001L.

Reverse transcription buffers suitable for reverse transcription reactions are known in the art. Illustrative buffers include, without limitation, the 10X Reverse transcription buffer marketed by Thermo Fisher can be used, included in kit number 4366597.

Tris-EDTA buffer 1X is 10 mM Tris-HCl, 1 mM disodium EDTA, pH 8.0 (such as Tris-EDTA buffer 1X sold by Interchim, Cat. # 58752A). Before addition to the RT premix, this buffer is diluted at 0.1 X. In a particular embodiment, TE buffer is in the RT premix at a 1268/1882 ratio (volume/total volume of the RT premix) in the RT premix.

The RT premix can be prepared by mixing the different reagents of said RT premix. In a particular embodiment, the RT premix is prepared by mixing the following compositions:
a dNTP mix comprising 100 mM of each dNTP;
10X reverse transcription buffer;
nuclease-free water;
Tris-EDTA, diluted to 0.1X;
primer specific of the miRNA of interest, diluted to 5X with Tris-EDTA 0.1X; and
primer specific of the control miRNA, diluted to 5X with Tris-EDTA 0.1X.

In a particular embodiment, the different compositions used in the preparation of the RT premix are mixed according to the following ratios (volume/total volume of the RT premix):
16/1882 of 100 mM each dNTP;
158/1882 of 10X reverse transcription buffer;
440/1882 of nuclease-free water;
634/1882 of Tris-EDTA, diluted to 0.1X;
317/1882 of a primer specific of hsa-miR-34a-5p, diluted to 5X with Tris-EDTA 0.1X; and
317/1882 of a primer specific of cel-miR-40-3p, diluted to 5X with Tris-EDTA 0.1X.

In yet another embodiment, the different compositions used in the preparation of the RT premix are mixed according to the following ratios (volume/total volume of the RT premix):
16/1882 of 100 mM each dNTP;
158/1882 of 10X reverse transcription buffer;
440/1882 of nuclease-free water;
634/1882 of Tris-EDTA, diluted to 0.1X;
317/1882 of the primer specific of hsa-miR-34a-5p of the TaqMan MicroRNA assay, kit number 4440888 (Thermo Fisher), diluted to 5X with Tris-EDTA 0.1X; and
317/1882 of the primer specific of cel-miR-40-3p of the TaqMan MicroRNA assay, kit number CCU001L (Thermo Fisher), diluted to 5X with Tris-EDTA 0.1X.

Below is provided a table which can be used as a non-limiting guideline to prepare a RT premix according to the invention, in different plate formats:

| Volume, μL | | | | | |
|---|---|---|---|---|---|
| (includes 10% for pipetting | | Cat. | 96 | 192 | 240 |
| losses) | Supplier | number | tests | tests | tests |
| 100 mM dNTPs | Thermo | Kit | 16 | 32 | 40 |
| | Fisher | 4366597 | | | |
| 10X Reverse transcription | Thermo | Kit | 158 | 317 | 396 |
| buffer | Fisher | 4366597 | | | |
| Nuclease free water | VWR | 733-2573 | 440 | 879 | 1098 |
| TE, diluted to 0.1X | Interchim | 58752A | 634 | 1267 | 1584 |
| hsa-miR-34a, diluted to | Thermo | Kit | 317 | 634 | 792 |
| 5X with TE 0.1X | Fisher | 4440888 | | | |
| Cel-miR-40, diluted to | Thermo | Kit | 317 | 634 | 792 |
| 5X with TE 0.1X | Fisher | CCU001L | | | |
| Total Volume per vial | | | 1882 | 3763 | 4702 |

Of course, larger volumes can be prepared for the preparation of more aliquots.

The RT premix of the invention is useful in the practice of a RT reaction. It can be prepared and stored at −20° C. or less. Advantageously, the inventors have shown that this RT premix can be stored at least 6 months in these conditions.

Quantitative Polymerase Chain Reaction Premix

A second aspect of the invention relates to a second composition, hereinafter referred to as a “qPCR premix” The qPCR premix of the invention comprises, or consists of:
a pair of primers suitable to amplify by qPCR a miRNA of interest;
a pair of primers suitable to amplify by qPCR a control miRNA, useful to normalize the quantity of the miRNA of interest; and
nuclease-free water.

In a particular embodiment, the miRNA of interest is miR-34, in particular hsa-miR-34a-5p whose nucleic acid sequence consists of the sequence shown in SEQ ID NO:1. Pairs of primers useful for use in a qPCR specific of hsa-miR-34a-5p are known in the art. For example, one skilled in the art can use the pair of primers of the TaqMan MicroRNA assay, kit number 4440888.

In a particular embodiment, the control miRNA is a miRNA which was spiked-in before the RT reaction, in particular cel-miR-40-3p. Pairs of primers useful for use in a qPCR specific of cel-miR-40-3p are known in the art. For example, one skilled in the art can use the pair of primers of the TaqMan MicroRNA assay, kit number 4440888.

The qPCR premix can be prepared by mixing the different reagents of said RT premix. In a particular embodiment, the qPCR premix is prepared by mixing the following compositions:
a pair of primers specific of the miRNA of interest, diluted to 20X with Tris-EDTA 0.1X;
a pair of premiers specific of the control miRNA, diluted to 20X with Tris-EDTA 0.1X; and
nuclease-free water.

In yet another embodiment, the qPCR premix is prepared by mixing the following compositions:
a pair of primers specific of hsa-miR-34a-5p, diluted to 20X with Tris-EDTA 0.1X;
a pair of primers specific of cel-miR-40-3p, diluted to 20X with Tris-EDTA 0.1X; and
nuclease-free water.

In a further embodiment, the qPCR premix is prepared by mixing the following compositions:
the pair of primers specific of hsa-miR-34a-5p of the TaqMan MicroRNA Assay, kit number 4440888 (Thermo Fisher), diluted to 20X with Tris-EDTA 0.1X;
the pair of premiers specific of cel-miR-40-3p of the TaqMan MicroRNA Assay, kit number 4440888 (Thermo Fisher), diluted to 20X with Tris-EDTA 0.1X; and
nuclease-free water.

In a particular embodiment, the different compositions used in the preparation of the qPCR premix are mixed according to the following ratios (volume/total volume of the qPCR premix):
158/527 of a pair of primers specific of hsa-miR-34a-5p, diluted to 20X with Tris-EDTA 0.1X;
26/527 of a pair of primers specific of cel-miR-40-3p, diluted to 20X with Tris-EDTA 0.1X; and
343/527 of nuclease-free water.

In a particular embodiment, the different compositions used in the preparation of the qPCR premix are mixed according to the following ratios (volume/total volume of the qPCR premix):
158/527 of the pair of primers specific of hsa-miR-34a-5p of the TaqMan MicroRNA Assay, kit number 4440888 (Thermo Fisher), diluted to 20X with Tris-EDTA 0.1X;
26/527 of the pair of primers specific of cel-miR-40-3p of the TaqMan MicroRNA Assay, kit number 4440888 (Thermo Fisher), diluted to 20X with Tris-EDTA 0.1X; and
343/527 of nuclease-free water.

Below is provided a table which can be used as a non-limiting guideline to prepare a qPCT premix according to the invention, in different plate formats:

| Volume, μL | | | | | |
|---|---|---|---|---|---|
| (includes 10% for pipetting | | Cat | 96 | 192 | 240 |
| losses) | Supplier | number | tests | tests | tests |
| TaqMan MicroRNA Assay | Thermo | Kit | 158 | 317 | 396 |
| hsa-miR34a primer, diluted | Fisher | 4440888 | | | |
| to 20x with TE 0.1X | | | | | |
| TaqMan MicroRNA Assay | Thermo | Kit | 26 | 53 | 66 |
| cel-miR40 primer, diluted | Fisher | 4440888 | | | |
| to 20x with TE 0.1X | | | | | |
| Nuclease free water | VWR | 733-2573 | 343 | 687 | 858 |
| Total Volume per vial | | | 527 | 1057 | 1320 |

Of course, larger volumes can be prepared for the preparation of more aliquots.

The qPCR premix of the invention is useful in the practice of a qPCR reaction. It can be prepared and stored at −20° C. or less. Advantageously, the inventors have shown that this qPCR premix can be stored at least 6 months in these conditions.

Method of Quantitatively Detecting a miRNA

The RT premix and the qPCR premix are useful in the practice of a RT-qPCR reaction.

Accordingly, the invention also relates to a method for quantitatively detecting a miRNA of interest in a RNA-containing test sample, comprising:
(a) a reverse transcription reaction, wherein RNA in the RNA-containing test sample is reversed transcribed into complementary DNA (cDNA), wherein the RT premix of the invention is mixed to a reverse transcriptase, a RNAse inhibitor and the RNA-containing test sample;
(b) a qPCR reaction to amplify the cDNA obtained at step (a), wherein the qPCR premix of the invention is mixed to a PCR master mix, to nuclease-free water and to the cDNA obtained at step (a).

The RNA-containing test sample is a sample from a mammal subject, in particular a human subject. In a particular embodiment, the RNA-containing test sample is a biological fluid of the subject, in particular a blood-derived sample, such as blood, serum or plasma. In yet another embodiment, the RNA-containing sample is total RNA from such a biological fluid. In yet another embodiment, the RNA-containing sample is small RNA from a biological fluid.

Step (a) is a reverse transcription reaction. Reverse transcriptase reaction reagents are readily available to those skilled in the art. For example, one can use the Multiscribe RT enzyme (50 U/μL) and the RNAse inhibitor (20U/μL ) from Thermo Fisher / Applied Biosystems (Cat. number 4366597).

The components of the RT reaction are mixed thoroughly before said reaction is conducted. The reaction tubes or plates can then be placed into a thermal cycler and incubated using standard cycling.

For example, the following settings can be used:

| Step | Time | Temperature |
|---|---|---|
| 1 cycle | 30 minutes | 16° C. |
| 1 cycle | 30 minutes | 42° C. |
| 1 cycle | 5 minutes | 85° C. |
| Hold | ∞ | 4° C. |

The RT reaction product, i.e. the cDNA, obtained after step (a), can be used immediately in a qPCR, or stored at −25 to −15° C. for up to one week.

Step (b) is then conducted from the cDNA obtained in step (a). A qPCR reaction mix is first prepared by mixing the qPCR premix of the invention to a PCR master mix and to the cDNA obtained in step (a).

The PCR master mix contains a DNA polymerase and reagents suitable to implement a PCR reaction. The skilled person knows many different materials and methods suitable to conduct a qPCR reaction, such as those marketed by Thermo Fisher.

A qPCR reaction also involves the use of means for detecting the formation of the amplified product, such as for the real-time detection of the amplified product. One can cite the use of the SYBR green dye which intercalates into double-stranded DNA, or the use of TaqMan probes which are fluorogenic single-stranded oligonucleotide probes that bind only the DNA sequence between the two primers used during the qPCR reaction. In a particular embodiment, the qPCR is based on the use of TaqMan probes. In a further particular embodiment, the qPCR reaction comprises the use of a probe specific of hsa-miR-34a-5p. In yet another embodiment, the qPCR reaction comprises the use of a probe specific of hsa-miR-34a-5p and of a probe specific of cel-miR-40-3p.

The components of the qPCR reaction are mixed thoroughly before said reaction is conducted. The reaction tubes or plates can then be placed into a thermal cycler and incubated using a suitable thermal cycle. For example, the following settings can be used:

| Step | Time | Temperature |
|---|---|---|
| 1 cycle | 10 minutes | 95° C. |
| 50 cycles | 15 seconds | 95° C. |
| | 60 seconds | 60° C. |

Methods of Diagnosis and Methods of Treatment

The method described above can be used to quantitatively detect a miRNA of interest in a sample of a subject.

In a particular embodiment, said quantitative detection can be part of a method for the diagnosis of a disease. In the context of the present invention, the expression “method for the diagnosis” denotes a method for the diagnosis, prognosis or monitoring of a disease, but also a method for evaluating the efficacy of a treatment against the disease.

The invention thus relates to a method for the diagnosis, prognosis or monitoring of a disease, wherein a miRNA of interest, such as hsa-miR-34a-5p is quantitatively detected according to the above method. This quantitative detection can be advantageously used in the diagnosis of non-alcoholic steatohepatitis (NASH), or for classifying a subject as a potential receiver of a treatment for NASH as described in PCT application W02017167934.

The invention further relates to a method for the diagnosis of a disease, comprising the determination of the level of a miRNA of interest in a biological sample of a subject, wherein the level of said miRNA is compared to the level of the same miRNA in a standard or positive control.

In a particular embodiment, the uses and methods of the invention comprise the comparison of the level of the miRNA determined from the biological sample to the level of a synthetic miRNA used as a standard or positive control.

### EXAMPLES

### Example 1: RNA Extraction

For the RNA extraction, 80 μl of Proteinase K and 230 μl of Lysis Buffer C were added to the plasma or serum sample—dispensed above the sample, then mixed by vortexing for 5 seconds before incubating at 37° C. for 15 minutes.

During this time, the MAXWELL RSC Cartridges (ref AS1680, Promega, USA) were prepared as described below. The cartridges were prepared shortly before adding the lysate:
place the cartridges to be used in the deck tray(s) with well #1 (the largest well in the cartridge) facing away from the elution tubes. Press down on the cartridge to snap it into position. Carefully peel back the seal so that all plastic comes off the top of the cartridge. Then place one plunger into well #8 of each cartridge, and an empty elution tube into the elution tube position for each cartridge in the deck tray.
add 50 μl of nuclease-free water to the bottom of each elution tube.
transfer all of the lysate (710 μl ) to well #1 (the largest well in the cartridge) of the MAXWELL RSC Cartridge, and add 10 μl of blue DNase I Solution to well #4 of the MAXWELL RSC Cartridge (well #4 contains yellow reagent).
the miRNA Plasma and Serum method are read on a MAXWELL RSC Instrument using the MAXWELL software. Touch Start to access the ‘Methods’ screen.
if sample eluates are not processed immediately, store the eluted RNA at −20° C. or −80° C. for longer storage time.

### Example 2: RT Reaction

The iscribe MicroRNA Reverse Transcription Kit (ref4366597, Applied Biosystems), includes
10x RT buffer
dNTP mix w/dTTP (100mM total)
RNAse inhibitor (20U/μl)
MULTISCRIBE RT enzyme (50U/μl).

For optimal performance of TAQMAN RNA Assays, the input RNA are RT inhibitors free, in a PCR-compatible buffer, RNAse free and non-denatured.

All reagents of the RT premix were kept on ice during preparation. RNAse Inhibitor and MULTISCRIBE Reverse Transcriptase were kept on a −20° C. cold block taken out from freezer just before use.

Mix for Controls

| Rounded Volume, μL | Supplier | Cat nbr | 1 test |
|---|---|---|---|
| RT Primers Premix | | | 17.8 |
| RNase inhibitor 20 U/μl | Applied | 4366597 | 0.2 |
| | Biosystems | | |
| MULTISCRIBE Reverse | Applied | 4366597 | 1.0 |
| Transcriptase, 50 U/μl | Biosystems | | |
| Total Volume per preparation | | | 20.9 |

The RT mix were vortexed gently and spined briefly before use. 19 μL of RT Mix were loaded in RT microplate well placed on ice.

For the positive controls, 5 μL were loaded in RT microplate. The plate were sealed with Microseal ‘B’ seal, homogenized by vortexing and the microplate were centrifuged 10 seconds at 500 g at room temperature. The RT plate was kept on ice for 5 minutes.

The Thermal cycler RT Program was the following:

| Step | Time | Temperature |
|---|---|---|
| 1 cycle | 30 minutes | 16° C. |
| 1 cycle | 30 minutes | 42° C. |
| 1 cycle | 5 minutes | 85° C. |
| Hold | ∞ | 4° C. |

The reaction plate was loaded into the thermal cycler, and the RT run was started.

### Example 3: qPCR Reaction

The TAQMAN Master Mix reagent was homogenized by swirling the bottle. qPCR primers and probes were thawed at room temperature and protected from light, then briefly centrifuged at 500 g, 30 sec.

RT plates with the cDNA that were stored frozen after the RT reaction were thawed on ice, vortexed and spun before use.

qPCR was also performed in duplex that includes the internal process control cel-miR-40.

| Rounded Volume, μL | Supplier | Cat nbr | 1 test |
|---|---|---|---|
| qPCR Premix (μL) | — | — | 5.0 |
| TaqMan Universal PCR Master Mix II | Applied | 4440040 | 10.0 |
| (μL) | Biosystems | | |
| Total Volume per preparation | | | 15.0 |

15 μl of qPCR reaction mix were transferred in each qPCR plate well.

5 μl of cDNA were added in qPCR plate.

The plate was sealed with Microseal vortexed (3 sec, speed 4) and centrifuged briefly.

Thermal-cyclers were in qPCR instruments area, regulated to 18-25° C.

The Bio-RAD CFX96 IVD thermal cycler was programmed as follows:

| Step | Time | Temperature |
|---|---|---|
| 1 cycle | 10 minutes | 95° C. |
| 50 cycles | 15 seconds | 95° C. |
| | 60 seconds | 60° C. |

The plate was loaded into the cycler and the run was started.

### Example 4: Premix Selection and Optimization for RT Primers

All necessary reagents were thawed on ice.

TE (Tris-EDTA, ref 58752A, Interchim) 0.1X was prepared extemporaneously with TE 1X and RNAse (Ribonuclease)-free water.

The RT primers were received in Thermo kit 4440888 (Thermo Fisher) were diluted to working solution at 5X prior to use with TE 0.1X and kept on ice.

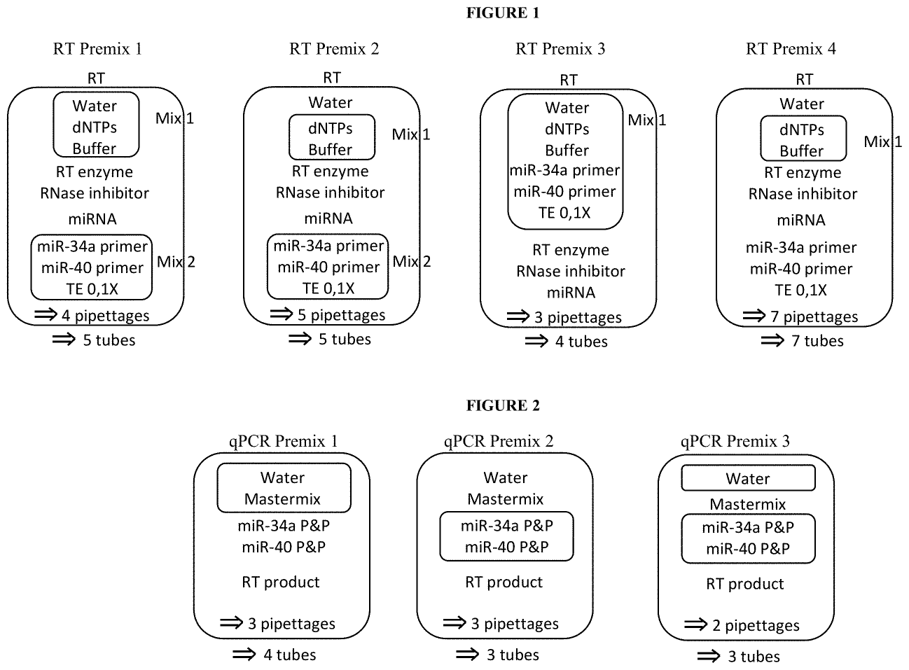

Several types of RT premixes were tested over the classical preparation, which contains 7 solutions to mix together. See FIG. 1.

Positive control C1, C2, C3 are samples prepared with miR-34a at respectively high (28.05 Cq corresponding to 5 to 8 in term of fold change expression), medium (30.92 Cq corresponding to around 1 in term of fold change expression) and low (33.71 Cq corresponding to 0.2 to 0.35 in term of fold change expression) levels (dosed by qPCR).

Positive controls were spiked with internal control miR-40 at 15.625 pM to have a Cq value between 28 and 29 Cq.

Briefly, mix 1, and eventually mix 2, were prepared. Then the mix were mixed with the other components of the kit to obtain the RT premix 1, 2, 3 or 4 and compared with the components mixed just before use (classical preparation), as recommended by the provider. All the components were mixed in proportions according to the provider.

The RT premixes 1 to 4 were tested for their stability at −20° C. over 6 months with 5 cycles freezing/thawing.

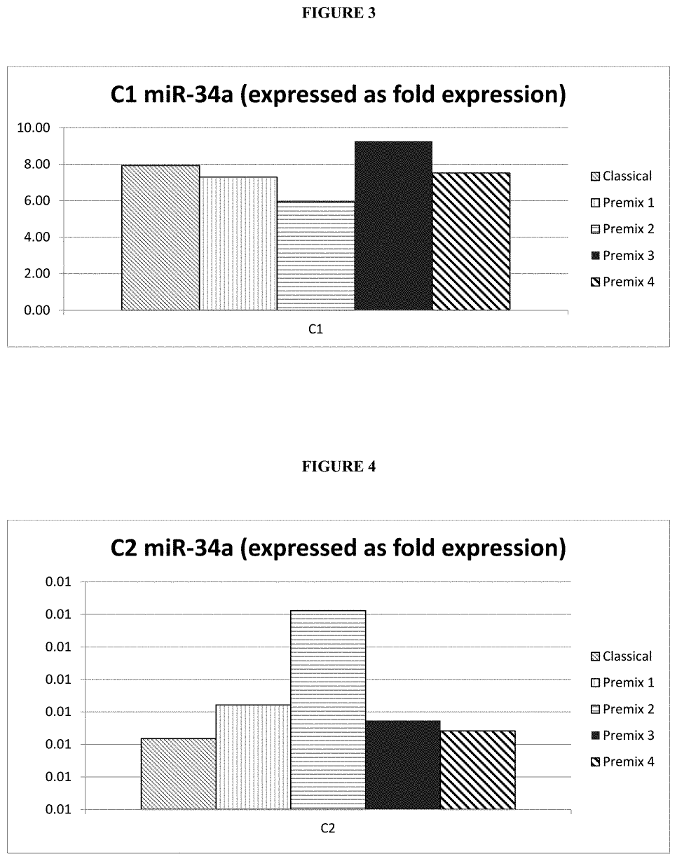

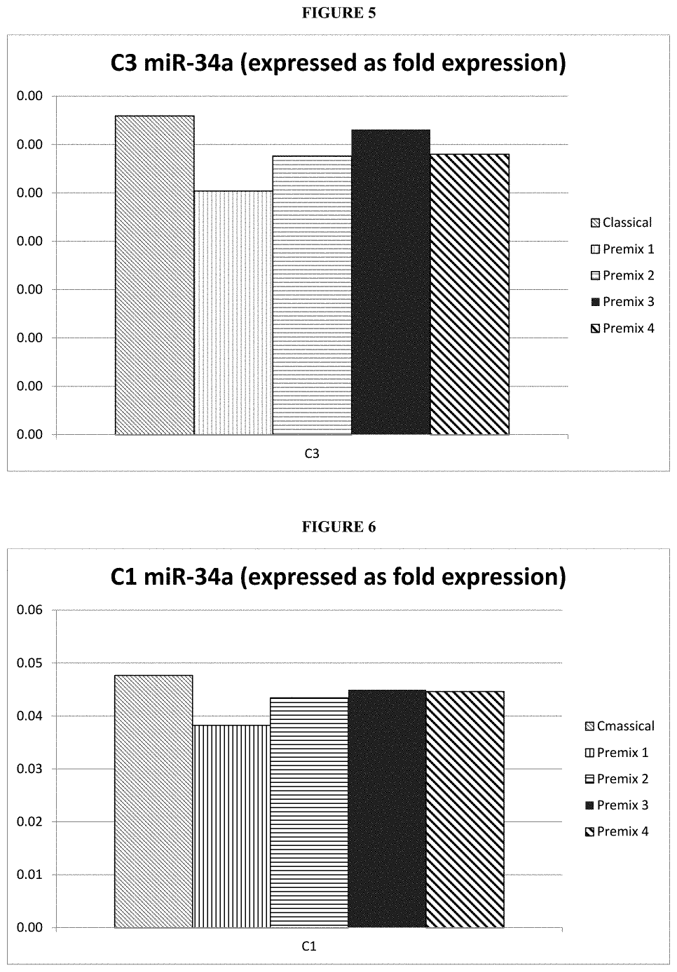

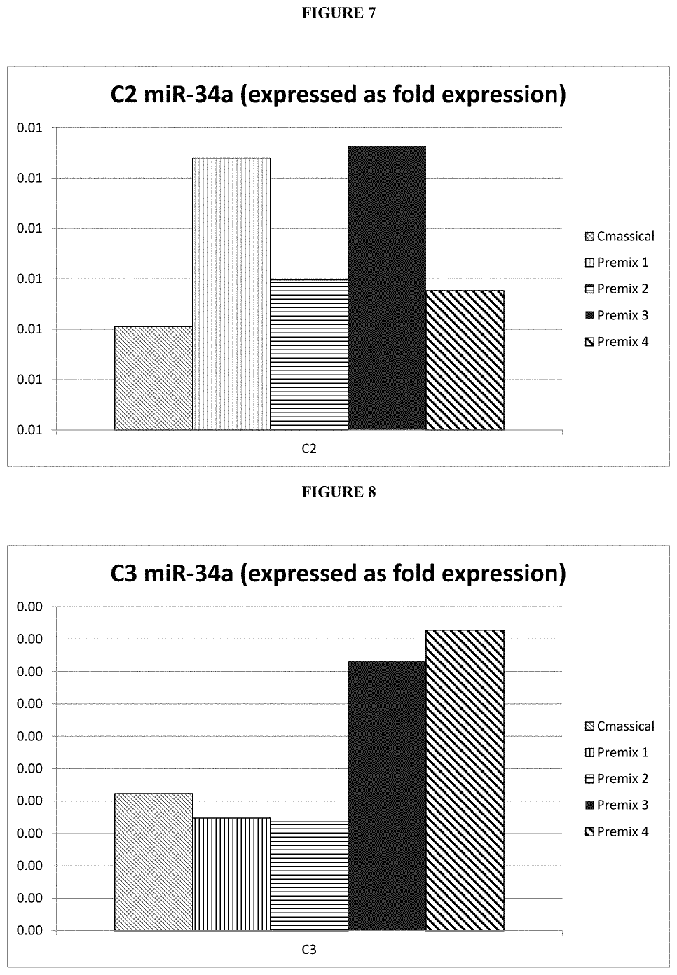

The 4 RT premixes were tested using miR34a positive controls (High, medium and low) and were compared to the classical preparation (FIGS. 3 to 5: after a 1 month-storage at −20° C., FIGS. 6 to 8: after a 6 month-storage at −20° C.).

Data are presented in fold expression of miR34a and showed that RT-Premix 3 gave the same data for the 3 controls as the classical preparation used in this experiment. RT-Premix 3 also allowed the minimal tubes to be used in RT compared to the other tested RT premixes.

### Example 5: qPCR Premix Selection

Four qPCR premixes were tested using miR34a positive controls (High, medium and low) and were compared to the classical preparation of the kit.

Several types of qPCR premixes were tested over the classical preparation, which contains 5 solutions to mix together according to the provider. See FIG. 2.

Positive controls C1, C2, C3 are samples prepared with miR-34a at respectively high (28.05 Cq=fold change between 5 and 8), medium (30.92 Cq=fold change around 1) and low (33.71 Cq=fold change between 0.2 and 0.35) levels (dosed by qPCR).

Positives controls were spiked with internal control miR-40 at 15.625 pM to have a Cq value between 28 and 29 Cq.

Briefly, water and mastermix (Premix 1) or miR-34a and miR-40 (Premix 2) or water, miR-34a and miR-40 (Premix 3). Then, each of the mixes of Premixes 1, 2 or 3 were mixed with the other components of the qPCR preparation depicted in FIG. 2. Premix 1, 2 or 3 were compared with the compounds mixed just before use (classical preparation), as recommended by the provider. All the components were mixed in proportions according to the provider.

The qPCR Premixes 1 to 3 were tested for their stability at −20° C. over 6 months with 5 cycles freezing/thawing.

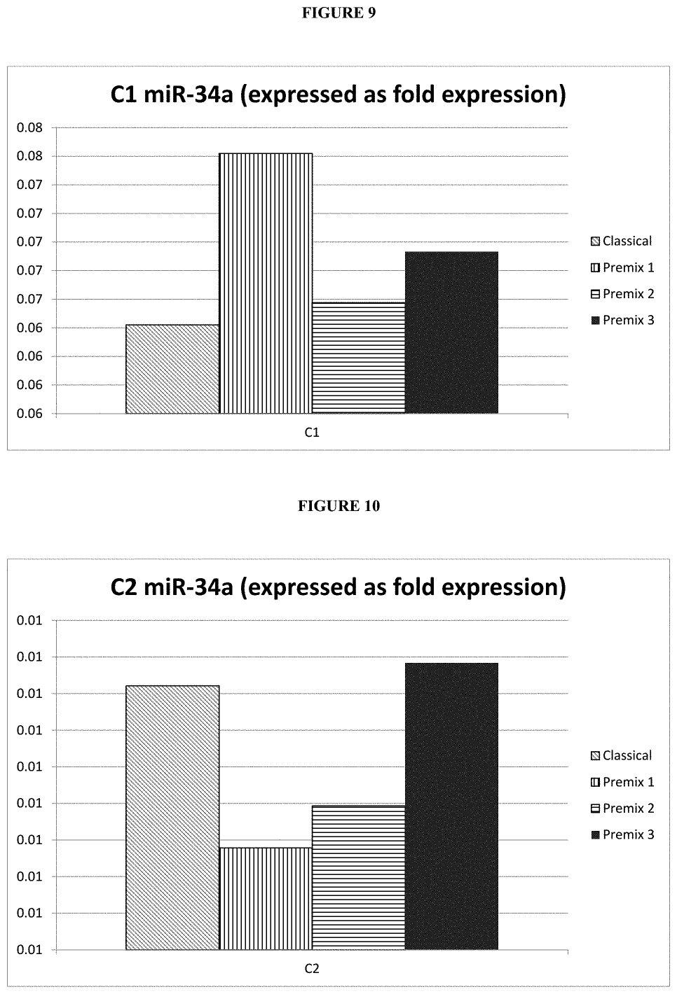

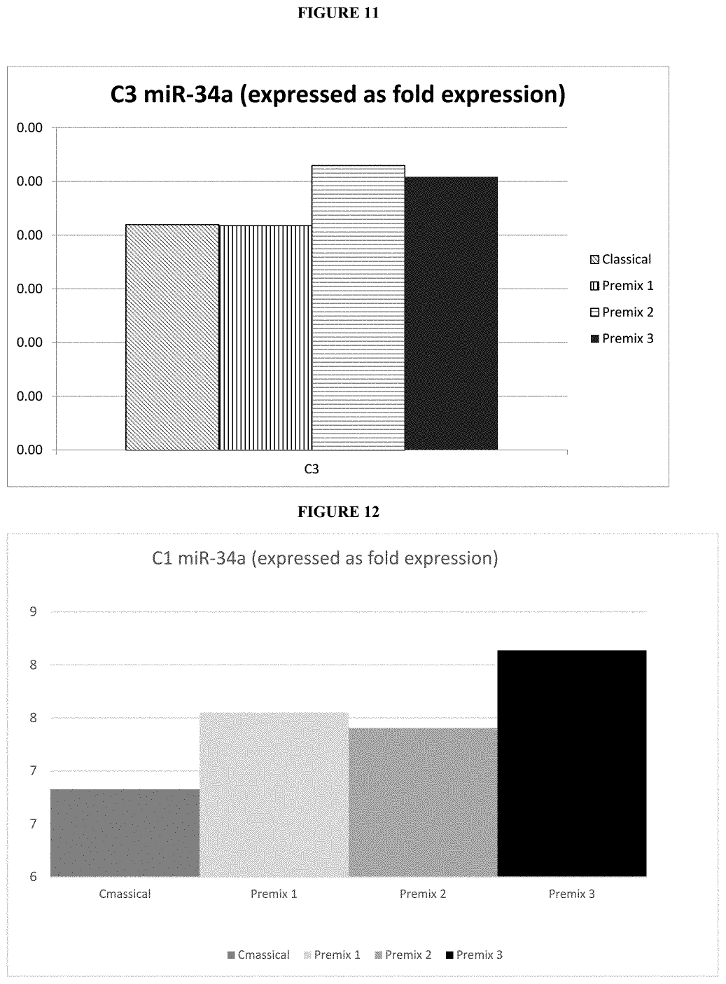

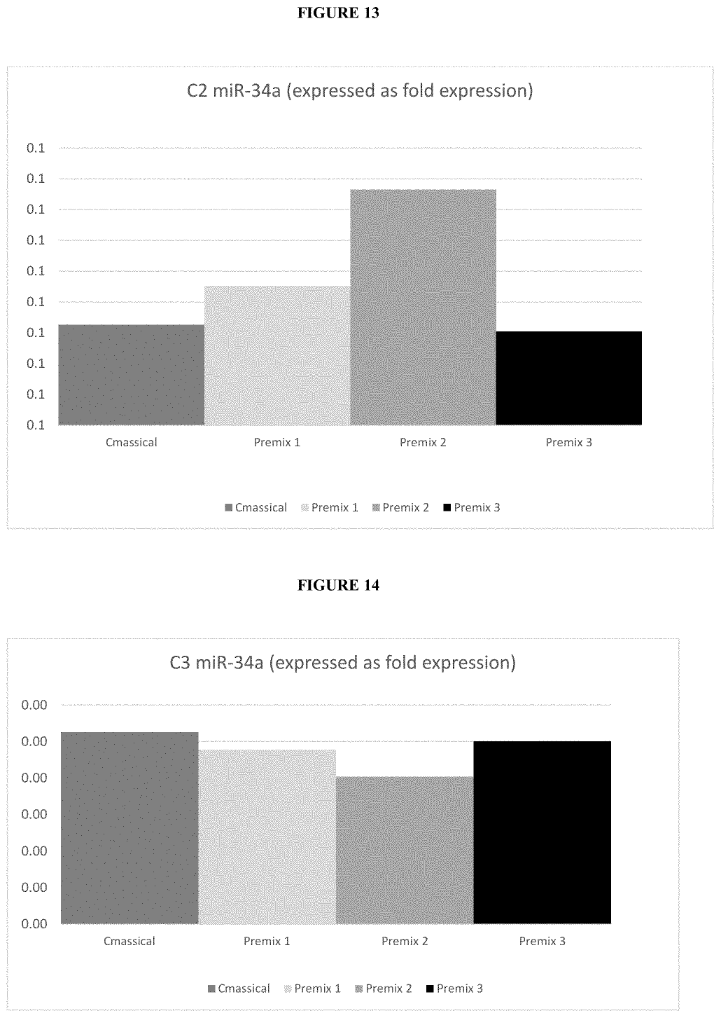

The 3 qPCR Premixes were tested using miR34a positive controls (High, medium and low) and were compared to the classical preparation (FIGS. 9 to 11: after a 1 month-storage at −20° C., FIGS. 12 to 14: after a 6 month-storage at −20° C.).

Data are presented in fold expression of miR34a and have shown that qPCR-Premix 3 gave the same data as the classical preparation for the 3 controls used in this experiment. qPCR Premix 3 also allowed the minimal tubes to be used in qPCR compared to the other tested RT premixes.

### Example 6: Final RT Premixes

All components were thawed on ice, vortexed briefly and spined before use.

Primers received with Thermo kit 4440888 were diluted to working solution at 5X prior to use.

The RT Primers Premix was prepared by mixing the reagents listed in the table below on ice.

Polypropylene low-binding tube was used to prepare the mixture.

The mixture was homogenized by gentle vortexing, and then aliquoted on ice for long term storage before use.

| Rounded Volume, μL | | | | | |
|---|---|---|---|---|---|
| (includes 10% for pipetting | | | 96 | 192 | 240 |
| losses) | Supplier | Cat nbr | tests | tests | tests |
| 100 mM dNTPs | Thermo | Kit | 16 | 32 | 40 |
| | | 4366597 | | | |
| 10X Reverse transcription | Thermo | Kit | 158 | 317 | 396 |
| buffer | | 4366597 | | | |
| Nuclease free water | VWR | 733-2573 | 440 | 879 | 1098 |
| TE, diluted to 0.1X | Interchim | 58752A | 634 | 1267 | 1584 |
| hsa-miR-34a, diluted to | Thermo | Kit | 317 | 634 | 792 |
| 5X with TE 0.1X | | 4440888 | | | |
| Cel-miR-40, diluted to | Thermo | Kit | 317 | 634 | 792 |
| 5X with TE 0.1X | | CCU001L | | | |
| Total Volume per vial | | | 1882 | 3763 | 4702 |

Premixes were stored at −20° C. until use. Stability was proven for at least 6 months. Frozen aliquots can be thawed and frozen up to 5 times.

### Example 7: Final qPCR Premixes

TAQMAN assays were thawed on ice, vortexed gently and spined briefly.

qPCR primers and probes were protected from light. Primers & probes received with Thermo kit 4440888 were diluted to working solution at 20X prior to use.

Premix was prepared by mixing the reagents listed in the table below on ice. Polypropylene low-binding tube was used to prepare the mixture.

The mixture was homogenized by vortexing and then aliquoted for long term storage before use.

| Rounded Volume, μL | | | | | |
|---|---|---|---|---|---|
| (includes 10% for pipetting | | | 96 | 192 | 240 |
| losses) | Supplier | Cat nbr | tests | tests | tests |
| TaqMan MicroRNA Assay | Thermo | Kit | 158 | 317 | 396 |
| hsa-miR34a primer, diluted | | 4440888 | | | |
| to 20x with TE 0.1X | | | | | |
| TaqMan MicroRNA Assay | Thermo | Kit | 26 | 53 | 66 |
| cel-miR40 primer, diluted | | 4440888 | | | |
| to 20x with TE 0.1X | | | | | |
| Nuclease free water | VWR | 733-2573 | 343 | 687 | 858 |
| Total Volume per vial | | | 527 | 1057 | 1320 |

Premixes were stored at −20° C. until use.

Stability was demonstrated for at least 6 months. Frozen aliquots can be thawed and frozen up to 5 times.

I claim:

1. A method for quantitatively detecting a microRNA (miRNA) in a sample, comprising:
(a) a reverse transcription (RT) reaction, wherein RNA in the RNA-containing test sample is reversed transcribed into complementary DNA (cDNA), wherein a RT premix is mixed to a reverse transcriptase, a RNAse inhibitor and the RNA-containing test sample;
wherein the RT premix comprises:
a deoxyribonucleotide trisphosphate (dNTP) mix containing dATP, dTTP, dCTP and dGTP;
a sequence-specific primer suitable to specifically prime the reverse transcription of the miRNA of interest to be quantitatively detected in a test sample;
a sequence-specific primer suitable to specifically prime the reverse transcription of a control miRNA, useful to normalize the quantity of the miRNA of interest;
a reverse transcription buffer;
Tris-EDTA buffer; and
nuclease-free water; and
(b) a quantitative polymerase chain reaction (qPCR) to amplify the cDNA obtained at step (a), wherein a qPCR premix is mixed to a PCR master mix, to nuclease-free water and to the cDNA obtained at step (a);
wherein the qPCR premix comprises:
a pair of primers suitable to amplify by qPCR a miRNA of interest;
a pair of primers suitable to amplify by qPCR a control miRNA, useful to normalize the quantity of the miRNA of interest; and
nuclease-free water.

2. The method of claim 1, wherein the test sample is a blood-derived sample from a human subject.

3. The method of claim 1, wherein the test sample is a serum sample from a human subject.

4. The method of claim 1, wherein the test sample is total RNA extract from a serum sample form a human subject.

5. The method of claim 1, wherein the miRNA of interest is hsa-miR-34a-5p.

6. The method of claim 1, wherein the control miRNA is cel-miR-40-3p.

7. The method of claim 1, wherein the control miRNA is cel-miR-40-3p spiked-in at a defined concentration in the test sample before step (a).
